(19) United States
(12) Patent Application Publication
(10) Pub. No.: US 2010/0063372 A1
(43) Pub. Date: Mar. 11, 2010

(54) SWEAT COLLECTION DEVICES FOR GLUCOSE MEASUREMENT

(76) Inventors: Russell O. POTTS, San Francisco, CA (US); James W. Moyer, San Francisco, CA (US); Hiroshi Yanazawa, Tokyo (JP); Irina Finkelshtein, San Jose, CA (US); Shuying Ye, Sendai (JP)

Correspondence Address: MORRISON & FOERSTER LLP, 755 PAGE MILL RD, PALO ALTO, CA, 94304-1018, US

(21) Appl. No.: 12/555,718

(22) Filed: Sep. 8, 2009

Related U.S. Application Data
(60) Provisional application No. 61/095,463, filed on Sep. 9, 2008.

(51) Int. Cl. A61B 5/1468

(52) U.S. Cl. 600/346

(57) ABSTRACT

Devices, methods, and kits for collecting sweat that has come to the surface of the skin are provided. The sweat may be collected for measuring sweat glucose levels. Because sweat glucose levels correlate to blood glucose levels, the provided devices, methods, and kits may be used by diabetic patients to non-invasively monitor blood glucose levels. Sweat collection devices may be attachable to the surface of the skin and may collect about one microliter or less of sweat. Because only a small, fixed volume of sweat may be collected, the sweat glucose level may be measured in a matter of minutes. Further, as a fixed volume of sweat is tested, inaccuracies due to estimates of the sweat volume being tested are less likely to cause an inaccurate glucose measurement.

### CROSS-REFERENCE TO RELATED APPLICATIONS

The present application claims priority under 35 U.S.C. §119(e) to U.S. Provisional Application No. 61/095,463 filed on Sep. 9, 2008, the disclosure of which is incorporated by reference herein in its entirety.

### FIELD OF INVENTION

The present application relates generally to glucose measurement from sweat that has come to the surface of the skin. More specifically, the present application relates to sweat collection devices attachable to the surface of the skin that are capable of collecting a known volume of sweat that is less than one microliter.

### BACKGROUND

The American Diabetes Association reports that approximately 7.8% of the population in the United States, a group of 23.6 million people, has diabetes, and that this number is growing at a rate of 12-15% per annum. The Association further reports that diabetes is the seventh leading cause of death in the United States, contributing to over 224,000 deaths per year. Diabetes is a life-threatening disease with broad complications, which include blindness, kidney disease, nerve disease, heart disease, amputation, and stroke. Diabetes is believed to be the leading cause of new cases of blindness in individuals between the ages of 20 and 74; approximately 12,000-24,000 people per year lose their sight because of diabetes. Diabetes is also the leading cause of end-stage renal disease, accounting for nearly 44% of new cases. Nearly 60-70% of people with diabetes have mild to severe forms of diabetic nerve damage which, in severe forms, can lead to lower limb amputations. People with diabetes are 2-4 times more likely to have heart disease and to suffer strokes than people without diabetes.

Diabetes results from the inability of the body to produce or properly use insulin, a hormone needed to convert sugar, starches, and the like into energy. Although the cause of diabetes is not completely understood, genetics, environmental factors, and viral causes have been partially identified.

There are two major types of diabetes: Type 1 and Type 2. Type 1 diabetes (also known as juvenile diabetes) is caused by an autoimmune process destroying the beta cells that secrete insulin in the pancreas. Type 1 diabetes most often occurs in young adults and children. People with Type 1 diabetes must take daily insulin injections to stay alive.

Type 2 diabetes is a metabolic disorder resulting from the body's inability to make enough, or properly to use, insulin. Type 2 diabetes is more common than Type 1 diabetes, accounting for 90-95% of diabetes. In the United States, Type 2 diabetes is nearing epidemic proportions, principally due to an increased number of older Americans and a greater prevalence of obesity and sedentary lifestyles.

Insulin, in simple terms, is the hormone that allows glucose to enter cells and feed them. In diabetics, glucose cannot enter the cells, so glucose builds up in the blood to toxic levels.

Diabetics having Type 1 diabetes are typically required to self-administer insulin using, e.g., a syringe or a pen with needle and cartridge. Continuous subcutaneous insulin infusion via external or implanted pumps is also available. Diabetics having Type 2 diabetes are typically treated with changes in diet and exercise, as well as with oral medications. Many Type 2 diabetics become insulin-dependent at later stages of the disease. Diabetics using insulin to help regulate their blood sugar levels are at an increased risk for medically-dangerous episodes of low blood sugar due to errors in insulin administration, or unanticipated changes in insulin absorption.

It is highly recommended by medical professionals that insulin-using patients practice self-monitoring of blood glucose (“SMBG”). Based upon the level of glucose in the blood, individuals may make insulin dosage adjustments before injection. Adjustments are necessary since blood glucose levels vary day to day for a variety of reasons, e.g., exercise, stress, rates of food absorption, types of food, hormonal changes (pregnancy, puberty, etc.) and the like. Despite the importance of SMBG, several studies have found that the proportion of individuals who self-monitor at least once a day significantly declines with age. This decrease is likely due simply to the fact that the typical, most widely used, method of SMBG involves obtaining blood from a capillary finger stick.

Because SMBG is so painful, measuring glucose levels in other ways that are non-invasive is desirable. Using sweat is attractive at least because it can be collected non-invasively and because sweat glucose level is correlatable to blood glucose level. However, collecting a sample of sweat that can be used to accurately measure the sweat glucose level is difficult.

Sweat may be excreted by sweat pores at a variable rate. For example, sweat production can vary significantly in the presence of physical or emotional stimulation such as activity level, stress, and heat. This variation may cause an inaccurate sweat glucose measurement as it can result in a fluctuation in the volume of sweat collected from the skin surface.

However, collecting a fixed volume of sweat is difficult as current collection devices may need to curve, bend, or twist to conform to a finger tip or other body surface, and the resulting deformation may change the volume of a container. Further, current collection devices are typically used to collect a large amount of sweat from the skin surface. For example, the Macroduct® sweat collection system by Wescor, Inc. (Logan, Utah) is capable of collecting up to sixty microliters of sweat regardless of the rate of sweat production. While using a large volume of sweat may decrease the effects of any variation in the collected volume, the amount of time required to collect the volume may increase.

Therefore, it would be desirable to provide devices and methods for collecting small, fixed volumes of sweat without being affected by a variable sweat rate in terms of the volume of sweat collected. Further, it would be desirable to provide methods for collecting a volume of sweat suitable for sweat glucose measurement from a skin surface. Finally, it would also be desirable to provide kits that can be used to monitor sweat glucose levels.

### SUMMARY

To reduce the likelihood of inaccuracies caused by estimating an unknown volume of sweat, a fixed volume of sweat may be collected from the surface of the skin each time the sweat glucose level is measured. A fixed-volume device for sweat collection generally comprises a channel layer, a container layer, and a vent layer. In some variations, the layers may be combined into a single layer and/or other layers may be added. The channel layer of the fixed volume device may contact the skin surface and direct sweat from the skin surface to an opening. On the skin surface, the sweat may be within or excreted from one or more sweat pores in contact with, or adjacent to, the channel layer. Typically, the container layer may be in fluid communication with an opening in the channel layer and may be in contact with the vent layer. The vent layer may be in contact with the container layer and may allow air to escape during sweat collection.

The container layer may partially define a container configured to contain less than about one-quarter microliter of sweat, about one-half microliter of sweat, about one microliter of sweat, about two microliters of sweat, about five microliters of sweat, about ten microliters of sweat, or any other suitable volume. In some embodiments, various properties of the sweat in the container may be measured using two or more electrodes disposed along the walls of the container.

The channel layer may have any number of channels to contact the skin for sweat collection. Upon contacting the skin surface, the channel layer may deform to contact as much skin as possible so that the channels may efficiently route sweat to the opening. The channel layer may have any suitable geometry or have any suitable dimensions. For example, the channel layer may have a thickness of about two hundred micrometers and the opening may have a diameter of less than about seven hundred micrometers. In some embodiments, the opening may have a diameter of greater than three hundred micrometers. The top side of the channel layer may define a bottom side of the container for holding the collected sweat. In these instances, the channel layer may or may not include one or more electrodes in contact with the container that is positioned to contact sweat within the container.

It may be desirable to induce sweat production to reduce the amount of time required to collect the fixed volume of sweat. For example, the channel layer may include a mechanism to deliver pilocarpine, other sweat-stimulating (i.e., diaphoretic) drugs, and/or heat to the skin.

The container layer may be positioned on top of or extend from the channel layer, and may have the same size and shape as the channel layer or be of a different size and/or shape. The channel layer may include at least one opening opposite the container layer to draw the sweat from the skin surface. The container layer may include a feature that defines at least one side of the container. The feature may be a hole, a well, an indentation, an absorbent portion, or the like. The thickness of the container layer may be selected based on one or more factors such as the shape of the container, the volume of the container, or rigidity required for the container to maintain its shape when the channel layer is deformed. For example, the container layer may have a thickness of approximately 100, 200, 500, 700, or 1,000 micrometers. Like the channel layer, the container layer may also comprise one or more electrodes positioned to contact sweat within the container. The electrodes may be used in conjunction with a measurement device to, for example, determine when the container contains the fixed volume of sweat and/or to measure the sweat glucose level.

The vent layer may be positioned on top of or extend from the container layer. In some variations, the functions performed by the vent layer may be performed by the container layer. The vent layer may reduce evaporation of sweat and/or provide an escape route for air within the container. In general, larger vents provide more fluid flow because the air can escape quickly but may allow more sweat to evaporate from the container. As such, the dimensions of the vents within the vent layer may be selected to provide a suitable balance between providing sufficient fluid flow and reducing the rate of evaporation from the container. In some embodiments, the vent layer has a thickness of approximately 100, 200, 500, 700, or 1,000 micrometers.

In some instances, the vent layer may include one or more electrodes in contact with the container that can be used to determine whether the container is filled and/or to measure the sweat glucose level. In various embodiments, an external surface of the vent layer comprises external electrodes that can be contacted by electrodes on a measurement device to measure the volume of sweat in the container and/or a sweat glucose level. Each external electrode may be connected to an internal electrode in contact with the container.

Methods for measuring a glucose level from sweat are also provided. In general, methods for measuring a glucose level from sweat comprise collecting a predetermined volume of sweat from skin using a skin patch and measuring the amount of glucose within the volume of sweat. The skin patch may be attached to any location on the body covered by skin. Typically, however, the skin patch is placed on a fingertip, hand, or forearm as these areas have a higher density of sweat glands, are easily accessible, and are currently used by diabetic patients for blood glucose testing. The skin patch may be a skin patch as described above or may be another skin patch that is configured to collect a predetermined volume of sweat. The predetermined volume of sweat may be less than about one-quarter microliter of sweat, about one-half microliter of sweat, about one microliter of sweat, about two microliters of sweat, about five microliters of sweat, about ten microliters of sweat, or any other suitable volume. Measuring the amount of glucose may comprise contacting the skin patch with a measurement device.

In some embodiments, the method also includes stimulating sweat production. Sweat production may be simulated chemically, e.g., by delivering pilocarpine to the skin surface. The pilocarpine may be wiped onto the skin surface prior to attachment of the skin patch. Sweat may also be stimulated by delivering heat or one or more other forms of energy to the surface of the skin. The patch itself may comprise a physical, chemical, or mechanical mechanism of inducing a local sweat response. For example, the patch may comprise pilocarpine, alone or with a permeation enhancer, or may be configured for iontophoretic delivery. Similarly, the patch may comprise one or more chemicals capable of inducing a local temperature increase, thereby initiating a local sweat response. In a like manner, the patch may also comprise one or more heaters for sufficient localized heating of the skin surface to induce an enhanced local sweat response.

The method for collecting sweat from the skin surface may additionally or alternatively include determining whether the volume of sweat collected is adequate prior to measuring the amount of glucose. In the sweat collection devices described here, the container may be configured to only collect up to the predetermined volume of sweat. Once the container is full, the sweat collection device may stop collecting sweat because there is no longer sufficient force to draw sweat into the container. Alternatively, by forming the vent layer from a hydrophobic material, the passage of sweat out of the container may be impeded. In some variations, the container may be defined by one or more hydrophilic surfaces while the vents may be defined by one or more hydrophobic surfaces. The determination that the container is full may be performed by providing an indicator, such as a dye, that changes the appearance of the skin patch, by a volume measuring device, or by an integrated device that also measures the sweat glucose level. In embodiments not comprising an indicator, the patient may remove the patch from the skin surface after an elapsed period of time with the assumption that the container should be full at that time.

Also described here are kits for collecting sweat. In some embodiments, the kits may also be used to measure a sweat glucose level. In general, a kit comprises one or more skin patches configured to collect a predetermined volume of sweat that is less than one microliter. The kit also includes a measurement device configured to measure an amount of glucose in the sweat, where the measurement is based on the predetermined volume. The skin patches may be configured for single use or for multiple uses (e.g., two to four uses). Each skin patch may have at least two electrodes in contact with the container that are connected to at least two corresponding external electrodes. The measurement device may comprise at least two electrodes configured to contact the skin patch at the external electrodes while the skin patch is attached to the skin surface. In some variations, the measurement device comprises an inlet configured to receive at least a portion of the skin patch.

### BRIEF DESCRIPTION OF THE DRAWINGS

FIG. 1a is a perspective view of a skin patch according to various embodiments.

FIG. 1b is a cross-sectional view of the skin patch of FIG. 1a according to various embodiments.

FIG. 1c is an exploded view of the various layers of the skin patch of FIG. 1a according to various embodiments.

FIGS. 2a through 2h depict a method for manufacturing a channel layer of a skin patch according to various embodiments.

FIGS. 3a through 3f depict a method for manufacturing a container layer of a skin patch according to various embodiments.

FIGS. 4a through 4f depict a method for manufacturing a vent layer of a skin patch according to various embodiments.

FIGS. 5a and 5b depict a method for molding the various layers of FIGS. 2a through 4f according to various embodiments.

FIG. 6 depicts a flow diagram for assembling the various layers of FIGS. 2a-4f according to various embodiments.

### DETAILED DESCRIPTION

Devices, methods, and kits for collecting a fixed volume of sweat that has come to a skin surface are provided. The volume of sweat may then be interrogated by a measurement device to provide a sweat glucose measurement as the sweat that has come to the skin surface via sweat pores contains an amount of glucose that correlates to the blood glucose level of a patient. For example, the fixed volume may be less than about one-quarter microliter of sweat, about one-half microliter of sweat, about one microliter of sweat, about two microliters of sweat, about five microliters of sweat, about ten microliters of sweat, or any other suitable volume. Additional information about collecting sweat from the sweat pores is provided in U.S. Patent Application Publication No. 2006/0004271 A1 entitled “Devices, Methods and Kits for Non-Invasive Glucose Measurement” by Thomas A. Peyser et al., which is hereby incorporated by reference herein in its entirety.

To determine when the fixed volume of sweat is collected, the skin patch may include a volume indicator. The volume indicator may include at least two electrodes which form a short circuit or an open circuit when the volume is collected. In other variations, the volume indicator may be chemical, mechanical, optical, or the like. The volume indicator may also operate concurrently or in conjunction with a measurement device.

The measurement device may be operated by coming into contact with the skin patch, for example, via optical or conductive measurement. The measurement device may, alternatively, receive the entire skin patch via an inlet. The measurement device may measure the sweat glucose level by any mechanism, including chemical, optical, and/or electromechanical mechanisms.

### Devices

In some embodiments, the sweat collection device is a skin patch, a chamber, a duct, or another device in fluid communication with one or more sweat pores. A sweat collection device may define a container having a fixed volume of, for example, less than one microliter. The container may be resistant to changes in shape or volume resulting from deformation, heat, or other conditions. In other instances, the container may comprise an absorbent material configured to absorb only a fixed amount of sweat.

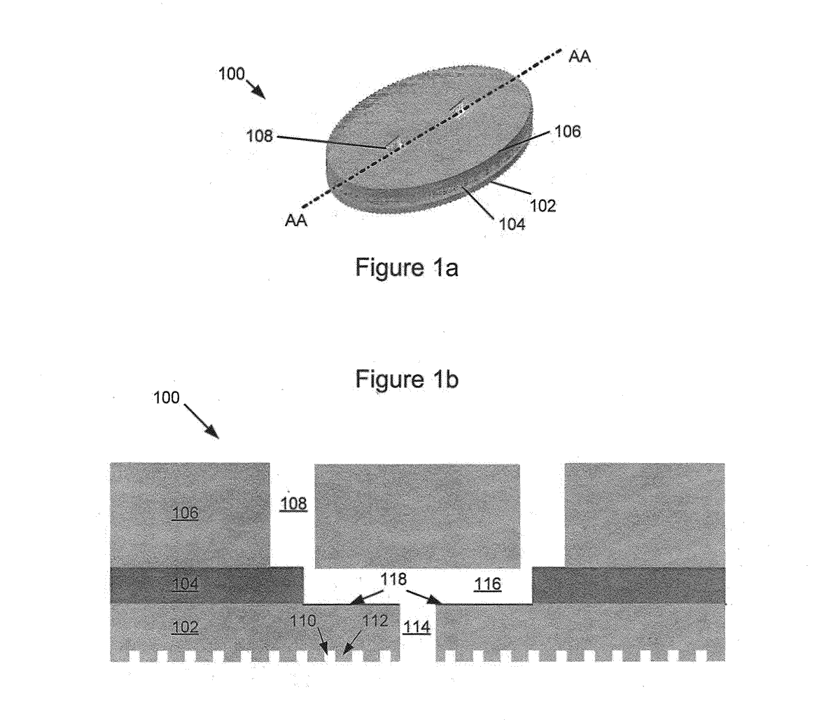

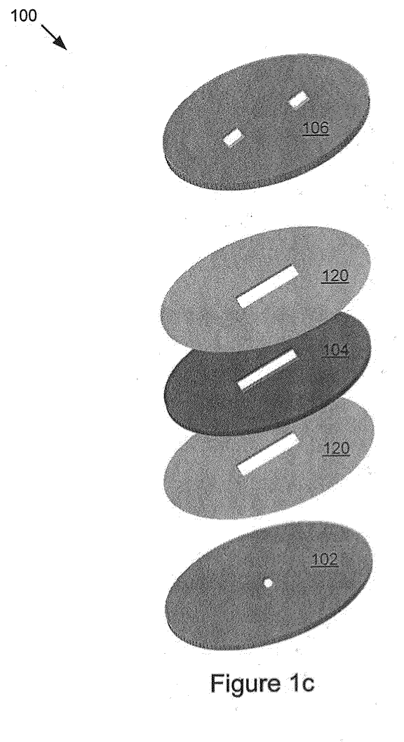

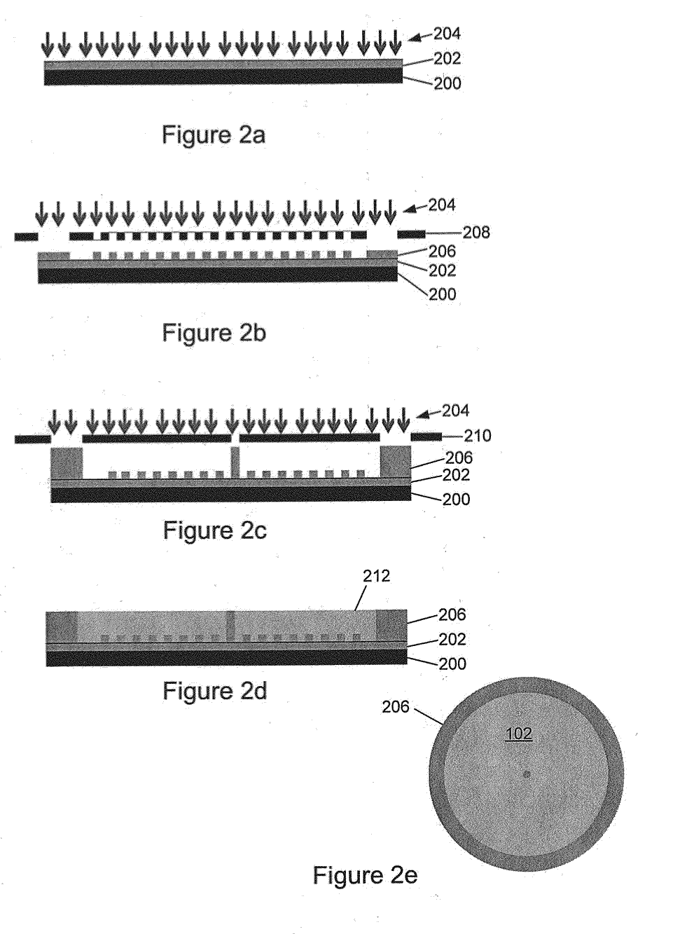

FIG. 1a is a perspective view of a skin patch 100 according to various embodiments. The skin patch 100 may comprise one or more layers to form or define a container for collecting sweat. The skin patch 100 may maintain contact with the skin using an adhesive or by any other suitable attachment mechanism (not shown) such as an elastic band, medical tape, or the like. In some embodiments, the skin patch 100 is configured to remain in contact with the skin for one minute, two minutes, five minutes, ten minutes, fifteen minutes, twenty minutes, thirty minutes, or longer depending on the amount of time required to collect a sufficient volume of sweat.

The skin patch 100 may have any shape (e.g., circular, as shown) and/or may be sized for a specific location on the body. For example, the skin patch 100 may be sized to attach to a fingertip. In other embodiments, the skin patch 100 may be sized and/or shaped to attach to another area of the hand, forearm, or other body location. The skin patch 100 may have a diameter of between about 10 mm and about 20 mm, about 20 mm and about 30 mm, about 30 mm and about 40 mm, and about 40 mm and about 50 mm. In some embodiments, the skin patch 100 may be another shape, such as a square, or triangle. In other embodiments, the skin patch 100 may be a fun shape such as a star, heart, dinosaur, or the like.

The skin patch 100, as shown, includes three layers of the same size. However, it should be understood that the skin patch 100 may contain a greater or lesser number of layers and that the one or more layers need not have a uniform size and shape. For example, a layer defining a container may be smaller than another layer to reduce the effects of deformation of the skin patch 100 on the volume of the container. The layers may or may not have a uniform thickness. For example, the layers may overlap, interlock, or otherwise interface with one another. The layers need not be continuous or contiguous. For example, a layer may be formed by one or more pieces fit together. In some embodiments, the layers may be fabricated using the same or different materials. In certain embodiments, one or more of the layers may be transparent, translucent, or opaque. The layers may be of different colors or the same color.

The channel layer 102 may be configured to contact the skin and to draw sweat from one or more sweat pores into a container. In some embodiments, the channel layer 102 may also be configured to stimulate sweat production. For example, the channel layer 102 may be coated, impregnated, or saturated with pilocarpine or another compound known to stimulate sweat production. Alternatively, the channel layer 102 may include depots or reservoirs containing the compound and that release the compound when in contact with the skin. In some embodiments, the channel layer 102 may include reservoirs for sweat-inducing compounds and/or micropumps for delivering the sweat-inducing compounds to the skin in contact with or adjacent to the channel layer 102. In certain embodiments, the channel layer 102 may include one or more channels and/or grooves to direct the sweat to the container as is discussed in greater detail in connection with FIG. 1c.

The skin patch 100 may also comprise a component to induce sweat by physical, chemical, or mechanical methods. For example, in one variation, the skin patch 100 comprises pilocarpine and a penetration or permeation enhancer to induce sweat chemically or pharmacologically. Similarly, heat may be applied to the skin to increase the sweat response.

While not shown in the figures, the skin patch 100 may also include at least one release liner. For example, a release liner on the bottom adhesive surface may protect the adhesive layer from losing its adhesive properties during storage and prior to use. Similarly, a release liner may be placed on top of the upper interface layer to protect the optical and/or electrical components contained therein. In some variations, no release liner is used and the interface layer is topped with a backing layer. In certain variations, the backing layer is made from a woven or non-woven flexible sheet, such as those known in the art of transdermal patches. In other variations, the backing layer is made from a flexible plastic or rubber.

To prevent the sweat collection device from collecting glucose from other sources, such as desquamation or diffusion, the channel layer 102 may comprise a sweat permeable membrane configured to collect only sweat being excreted by the sweat pores in contact with the channel layer 102. Examples of sweat permeable membranes include hydrophobic materials such as petrolatum, paraffin, mineral oils, silicone oils, vegetable oils, waxes, a liquid polymer coating such as the SILGARD® silicon polymer, an inorganic membrane such as the ANOPORE® inorganic membranes, a membrane filter such as the Whatman NUCLEOPORE® polycarbonate track-etch membrane filters, and the like.

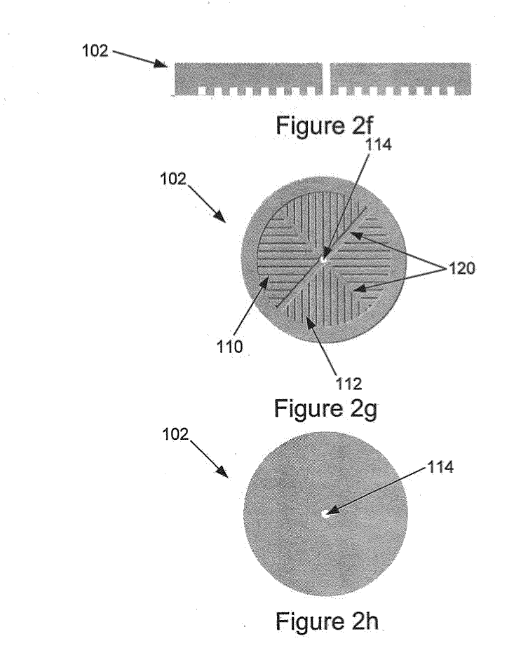

Alternatively or additionally, the channel layer 102 may be fabricated using one or more hydrophobic materials. Hydrophobic materials may be used to repel sweat from the bottom surface of channel layer 102 through an opening and into a hydrophilic container within the skin patch 100. The hydrophobic material may be selected based on flow properties, optical properties, conformability, viscoelasticity, flammability, toxicity, inertness, and/or the like. An example of a hydrophobic material that can be used in the manufacture of the channel layer 102 is polydimethylsiloxane (PDMS). One process that may be used to fabricate the channel layer 102 is discussed in greater detail in connection with FIGS. 2a through 2h.

The container layer 104 is configured to at least partially define a container that collects a fixed volume of sweat. The container layer 104 may be fabricated using a rigid material to prevent deformation and/or change in volume of the container. The fixed volume may be selected based on the sensitivity of the glucose detector and/or the amount of time required for collection of the volume of sweat. In some embodiments, the container layer 104 may be fabricated using polymethylmethacrylate (PMMA). The container layer 104 may be hydrophilic or may be made of any other suitable material. In some embodiments, the channel layer 102 and/or the container layer 104 may comprise one or more micropumps configured to pump the sweat into the container from the skin in contact with the skin patch 100.

The vent layer 106 may be fabricated using PDMS, PMMA, and/or any other suitable material or materials. It may be desirable to fabricate the vent layer 106 of a hydrophobic material to limit or prevent evaporation from the hydrophilic container layer 104 especially, for example, once the container is filled. The vent layer 106 comprises at least one vent 108 connecting the container to an external surface. The vent or vents 108 may comprise one or more lumens through the vent layer 106. In some embodiments, the inner surface of the vents 108 may be hydrophobic. The vents 108 may have any suitable cross-sectional geometry. For example, a vent 108 may have a circular, rectangular, regular, irregular, or any other suitable cross-sectional geometry. In addition, the vents 108 may be vertical, angled, curved, stepped, or any combination thereof. The vents 108 may or may not be configured to change shape if the skin patch 100 is deformed.

The vents 108 may provide an escape for air trapped in the skin patch 100 when it is applied to the skin and may facilitate the fluid flow of the skin patch 100. As the vents 108 become larger, however, the sweat in the container is more likely to evaporate. Thus, the size of the vents 108 may be balanced between being large enough to provide sufficient fluid flow and small enough to prevent a significant amount of sweat from evaporating. The vents 108 may completely or partially overlap a portion of the container. Partially overlapping the vents 108 may prevent some evaporation.

FIG. 1b is a cross-sectional view of the skin patch 100 taken along line AA-AA of FIG. 1a according to various embodiments. The skin patch 100, as shown, may have a total height of between about 500 and about 1500 micrometers. In some embodiments, the total height may be between about 500 and about 700 micrometers, about 700 and about 900 micrometers, about 900 and about 1100 micrometers, about 1100 and about 1300 micrometers, and about 1300 and about 1500 micrometers. In some embodiments, the total height of the skin patch 100 may be about 900 micrometers. The total height of the skin patch 100 may be determined based on manufacturing cost, durability, ease of use, materials used to manufacture the skin patch 100, or any other suitable factor.

As is shown in the cross-sectional view, the channel layer 102 may comprise a plurality of microchannels 110 defined by channel walls 112. The microchannels 110 are positioned to direct sweat that has come to the surface of the skin to an opening 114. The dimensions of the channels may be adjusted based on a desired collection rate and efficiency. In some embodiments, the channel layer 102 may have a thickness of between 100 and 500 micrometers. For example, the thickness of the channel layer 102 may be between 100 and 200 micrometers, between 200 and 300 micrometers, between 300 and 400 micrometers, or between 400 and 500 micrometers. As an example, the thickness of the channel layer 102 may be about 215 micrometers. The microchannels 110 may each have a width of about 10 micrometers to about 100 micrometers and/or a depth of about 2 micrometers to about 50 micrometers. As an example, the microchannels 110 may each have a width of about 38 micrometers and/or a depth of about 15 micrometers. The channel walls 112 may each have a width of about 20 micrometers to about 250 micrometers. As an example, the channel walls 112 may each have a width of about 80 micrometers.

The opening 114 may be located at or near the center of the channel layer 102 to provide fluid communication between the skin surface and a container 116. In some embodiments, the channel layer 102 may include more than one opening. In certain embodiments, a surface of the opening 114 may be coated with one or more hydrophilic materials to attract the sweat from the microchannels 110. Alternatively or additionally, a microfluidic pump may be used to transport the sweat from the skin in contact with the channel layer 102 through the opening 114. To direct the sweat towards the opening 114 and into the container 116, the surface of the channel layer 102 may be hydrophobic. In some embodiments, the channel layer 102 may be fabricated using a hydrophobic material such as PDMS. Alternatively or additionally, the channel layer 102 may be at least partially coated with a hydrophobic material.

As shown in FIG. 1b, the container layer 104 may at least partially define a container 116 configured to collect and hold a fixed volume of sweat. The fixed volume of sweat may be relatively small. In some embodiments, the fixed volume of sweat is less than one microliter, less than 0.75 microliter, less than 0.5 microliter, less than 0.25 microliter, or less than 0.1 microliter. In certain embodiments, the container layer may have a thickness of approximately 100, 200, 500, 700, or 1,000 micrometers. To maintain the fixed volume, the container 116 may be rigid enough to retain its shape when the skin patch 100 is deformed. For example, the container layer 104 may be fabricated from a rigid material such as PMMA.

In the embodiments shown, the container 116 is rectangular in shape. However, the container 116 may be of any suitable shape. For example, the container 116 may be cylindrical. As shown, the depth of the container 116 is approximately equal to the thickness of the container layer 104. In some embodiments, the depth of the container 116 may be different from the depth of the container layer 104 depending on, for example, the geometry of the channel layer 102 and the vent layer 106. The container 116 may be shallower or deeper based on the shape of the container 116 and/or the fixed volume of sweat to be collected. In some embodiments, the container 116 may be shallower. In other embodiments, the container 116 may be deeper (e.g., to reduce sweat evaporation).

In the embodiments shown, the container 116 is defined by the channel layer 102, the container layer 104, and the vent layer 106. The bottom of the container 116 is defined by a top side of the channel layer 102. The sides of the container 116 are defined by the container layer 104. The top of the container 116 is defined by the vent layer 106.

In alternative embodiments, the skin patch 100 may not include a vent layer 106. In these embodiments, sweat may be drawn into the container 116 using, for example, a pressure gradient. For example, the container 116 may be evacuated prior to application to the skin or a suction device may be coupled to the container 116 to provide a pressure gradient.

Because the channel layer 102 may be hydrophobic, its top surface may be at least coated with a hydrophilic coating 118 to attract the sweat into the container 116. Further, the opening 114 may also be coated with one or more hydrophilic materials. Hydrophilic materials that may be used include, but are not limited to, glass, 2-hydroxethyl methacrylate (HEMA), poly(oxyethylene) (POE), silicon dioxide, poly(ethylene glycol) (PEG), and polyacrylamide. In some variations in which the channel layer 102 is formed of PDMS, surface modifications of the PDMS may be performed by, for example, oxygen plasma treatments, or UV-mediated grafting.

The container 116 may include a volume indicator configured to indicate when the container 116 has collected the predetermined volume of sweat. The volume indicator may be electrical, mechanical, optical, chemical, or the like. For example, the top side of the container 116 may be coated with a sweat-sensitive or water-sensitive dye that changes color when the container 116 is full.

Alternatively, the container 116 may include electrodes that can provide a conductive path through the fixed volume reservoir when the reservoir is full. Changes in resistance or conductance at the top of the reservoir may be measured to determine when the container 116 has collected the fixed volume of sweat. The modest power required to drive a current through the circuit described here may be provided by an inductive coupling mechanism enclosed within a measurement device, a plastic battery, or the like.

Optical transmission may also be used to determine when the container 116 is filled. When on a skin surface, the skin patch 100 fills with sweat that has passed through the opening 114 and into the container 116. An optical transmission path is established with the container 116. In this way, the volume within the container 116 may be determined by a change in optical transmission (e.g., at the top of the container 116). An optical fiber path may connect an optical source on one side of the skin patch 100 with an optical detector on the other. Changes in the measured transmission may indicate whether the fluid volume in the container 116 has reached a maximum. Power for the optical source and detector may be included in a measurement device.

Optical reflection may also be used to determine when the container 116 is filled. A transparent plate (not shown) may be located on the top of the container 116 and may comprise at least a portion of the vent layer 106. This plate may have an optical index of refraction close to that of sweat (about 1.33). Incident light may illuminate the interface between the container 116 and the plate. If the container is not full, the reflected light may have a high intensity because the optical index difference between the plate and air (which has an optical index of refraction of about 1.0) is high. If the container 116 is full, however, the reflected light has a low intensity because the optical index difference between the plate and sweat is low (both have an optical index of refraction of about 1.33). Thus, the drop in reflected light intensity may be used as an indicator that the container 116 is full. An optical source and detector may be included in a measurement device and the skin patch 100 can be interrogated via an optical interface.

The container 116 may comprise one or more enzymes used to measure glucose, such as glucose oxidase. The enzyme or enzymes may be deposited within the container so that the sweat contacts the enzyme or enzymes. In some embodiments, the container 116 may be adjacent to one or more wells or deposits of the enzyme or enzymes. One or more surfaces, including electrodes and/or optical components, may include or be coated with the enzyme or enzymes.

FIG. 1c is an exploded view of the various layers of the skin patch of FIG. 1a according to various embodiments. As previously discussed, the skin patch 100 may comprise a channel layer 102, a container layer 104, and a vent layer 106. The layers may be adhered, glued, fastened, interlocked, welded, or otherwise suitably coupled together. As shown in FIG. 1c, in some variations, one or more layers of the skin patch 100 may be adhered together using an adhesive 120. In certain variations, one or more layers of the skin patch 100 may include fasteners, slots, tabs, latches, or the like. In some embodiments, the layers of the skin patch 100 may include one or more interlocking features.

The adhesive 120 may comprise a permanent or temporary adhesive and may be selected based on the materials used to fabricate the layers. The adhesive 120 between the channel layer 102 and the container layer 104 may be the same as or different from the adhesive 120 between the container layer 104 and the vent layer 106. For example, one of the adhesives may be a temporary adhesive while the other may be a permanent adhesive. The adhesive 120 may be activated by heat, pressure, the presence of a solute, or any other appropriate bonding technique. In some embodiments, the adhesive 120 may comprise an acrylic adhesive such as those available from Cemedine Co., Ltd., Japan or a silyl urethane adhesive such as those available from Conishi Co., Ltd., Japan.

The above described devices are described herein for the purposes of illustration and are not intended to be limiting. Alternative and additional embodiments may be apparent to those skilled in the art.

### Methods of Manufacture

Various methods may be used to manufacture the skin patch 100. In some embodiments, the layers are each manufactured separately and later assembled. In other embodiments, the layers may be assembled during manufacture, for example, one layer may be fabricated directly on top of or beneath another layer. The layers may be cut, molded, or otherwise fabricated. In some embodiments, micro-molding techniques and/or photolithography techniques may be used. In other embodiments, other suitable techniques, such as micro-machining, may be used.

In some embodiments, the layers may be treated or modified prior to being assembled. The layers may, for example, be at least partially modified to change the hydrophobic or hydrophilic nature of the materials used. For example, a hydrophilic coating may be applied to at least a portion of a layer fabricated from a hydrophobic material such as PDMS. Hydrophilic materials that may be used include, but are not limited to, glass, 2-hydroxethyl methacrylate (HEMA), poly(oxyethylene) (POE), silicon dioxide, poly(ethylene glycol) (PEG), and polyacrylamide. Surface modifications of PDMS may also be performed by, for example, oxygen plasma treatments and/or UV-mediated grafting.

Additionally, one or more features may be added to the layers. These features may include electrodes, dyes, a transparent plate, an enzyme coating or deposit (e.g., glucose oxidase), or the like. The electrodes may be positioned so as to be in contact with a portion of the container 116 once the skin patch 100 is assembled. The electrodes may be electrically coupled to one or more leads or external electrodes that can be accessed by a volume indicator or a measurement device. Similarly, a dye, such as a visible dye or a fluorescent dye, may be coated or applied to a portion of at least one of the layers. The dye may be configured to react in response to the presence of sweat. In some instances, a dot of dye may be applied to a top side of the container 116 such that the dye will diffuse along the top of the container, changing the shape of the dot, when the container 116 is full.

An exemplary method for generating the skin patch 100 is described below for the purposes of illustration only. It should be understood that the methods may be performed in another order, performed in parallel, and/or steps may be added and/or combined. Further, depending on the specific circumstances at the time of fabrication and the materials used, temperatures, times, materials, and techniques may be changed.

### Example

FIGS. 2a through 2h depict a method for manufacturing a channel layer 102 of a skin patch 100 according to various embodiments. As depicted in FIGS. 2a through 2c, a release layer 202 is generated. As shown in FIG. 2a, to form the release layer 202, a negative-tone UV light-sensitive photoresist, such as an SU-8 dry film, of about 50 micrometers thick may be laminated on a four inch silicon wafer 200 under a vacuum using a laminating machine (e.g., VTM-150M, Takatori Corporation, Japan) and then exposed under UV light 204 (22 mw/cm²) for about 20 seconds.

Next, as shown in FIG. 2b, to form a mold 206, an SU-8 dry film of about 15 micrometers may be laminated on the release layer 202. This layer may be exposed to UV light 204 through a mask 208 that defines the plurality of the channels of the channel layer 102 for about 18 seconds. After exposure, the wafer 200 may be baked on a hotplate at about 65° C. for one minute, and then at about 95° C. for five minutes. Next, the wafer 200 may be developed in a standard developing solution (available from, e.g., Nippon Kayaku Co., Ltd.) for one minute under stirring and dressed in a fresh developer for 15 seconds, and then rinsed using isopropyl alcohol (IPA) for about thirty seconds and de-ionized (DI) water for about three minutes followed by drying using nitrogen gas. To fabricate a rigid mold, the wafer 200 may be baked on the hotplate at 120° C. for about ten minutes.

As shown in FIG. 2c, to complete the mold 206, an SU-8 layer of about 200 micrometers thick may be formed by laminating the SU-8 film of about 50 micrometers thick four times as described in connection with FIG. 2b. The wafer 200 may be exposed under UV light 204 through another mask 210 for about eighty seconds. The mask 210 may define the location of the opening 114. The process of developing, rinsing, and baking may be performed as described above but the time for development for an SU-8 layer of 200 micrometers thick may be about 20 minutes. As a result, a mold 206 of the channel layer 102 may be formed.

Next, a PDMS prepolymer mixture 212 may be poured onto the mold 206 as depicted in FIG. 2d. A PDMS prepolymer mixture may be obtained by mixing a curing agent (e.g., KE-106, Shin-Etsu Chemical Co. Ltd, Japan) with PDMS prepolymer in a 1:10 volume ratio. After agitating the resulting PDMS prepolymer mixture 212 using a stir stick, the PDMS prepolymer mixture 212 may be degassed in a vacuum container for about one hour. The mold 206 may be heated on a hot plate for curing. After the mold 206 has been cured, it may be peeled off from the release layer 202 along with the PDMS.

The mold 206 may be peeled or otherwise removed from the channel layer 102, leaving the channel layer 102 behind, as depicted in FIGS. 2f through 2h. FIG. 2f depicts a cross section of the channel layer 102 as discussed herein.

FIG. 2g depicts the bottom side of the channel layer 102. The bottom side of the channel layer 102 may comprise a plurality of microchannels 110 defined by channel walls 112. In the depicted embodiments, the channel layer 102 comprises two main channels 120. The two main channels 120 may provide fluid communication with the opening 114. The main channels 120 bisect the channel layer 102 but other geometries may be used. The main channels 120 may have a depth and/or thickness larger than the depth and/or thickness of the microchannels 110. For examples, the depth and/or thickness of the main channels 120 may be 1.1, 1.2, 1.5, 1.8, 2.0, 3.5, 5.0, or 10.0 times the depth and/or thickness of the microchannels 110.

FIG. 2h depicts the top side of the channel layer 102. The top side includes the opening 114 and may be coated with a hydrophilic material. In some embodiments, the top side may have embedded therein one or more electrodes, chemical detectors, and/or mechanical indicators that form part, or all, of a volume indicator configured to indicate when the container 116 is full.

In some embodiments, the top side of the channel layer 102 and/or the interior surface of the channel layer 102 that defines the opening 114 may be coated with a hydrophilic material. The hydrophilic material may aid the transportation of the sweat from the skin surface to the container 116 by attracting water in the sweat. The hydrophilic material may be sprayed, painted, dropped, impregnated, or otherwise applied to the channel layer 102 by any appropriate means. In some embodiments where the channel layer 102 is fabricated using PDMS, which is hydrophobic, the hydrophilic material may comprise FogClear® hydrophilic gel (Unelko Corp., Scottsdale, Ariz.).

In alternative embodiments, the PDMS may be treated according to methods known to those skilled in the art. These techniques may include coating the PDMS with glass, 2-hydroxethyl methacrylate (HEMA), poly(oxyethylene) (POE), silicon dioxide, poly(ethylene glycol) (PEG), and polyacrylamide. Surface modifications of the PDMS may also be performed by, for example, oxygen plasma treatments, or UV-mediated grafting. Various hydrophilic treatments for PDMS using these techniques are disclosed in, e.g., Abate et al., “Glass coating for PDMS microfluidic channels by sol-gel methods,” Lab Chip, 2008, 8, 516-518, 20 Feb. 2008; Bodas et al., “Formation of more stable hydrophilic surfaces of PDMS by plasma and chemical treatments,” Microelectronic Engineering 83 (2006) 1277-1279, 23 Feb. 2006; Bodas et al., “Fabrication of long-term hydrophilic surfaces of poly(dimethyl siloxane) using 2-hydroxy ethyl methacrylate,” Sensors and Actuators B 120 (2007) 719-723, 2 May 2006; Delamarche et al., “Microcontact Printing Using Poly(dimethylsiloxane) Stamps Hydrophilized by Poly(ethylene oxide) Silanes,” Langmuir 2003, 19, 8749-8758, 11 Sep. 2003; Eddington et al., “Thermal aging and reduced hydrophobic recovery of polydimethylsiloxane,” Sensors and Actuators B 114 (2006) 170-172, 4 Jun. 2005; He et al., “Preparation of Hydrophilic Poly(dimethylsiloxane) Stamps by Plasma-Induced Grafting,” Langmuir 2003, 19, 6982-6986, 19 Jul. 2003; Hellmich et al., “Poly(oxyethylene) Based Surface Coatings for Poly(dimethylsiloxane) Microchannels,” Langmuir 2005, 21, 7551-7557, 6 Jul. 2005; Hu et al., “Surface-Directed, Graft Polymerization within Microfluidic Channels,” Anal. Chem. 2004, 76, 1865-1870, 3 Mar. 2004; Hu et al., “Tailoring the Surface Properties of Poly(dimethylsiloxane) Microfluidic Devices,” Langmuir 2004, 20, 5569-5574, 25 May 2004; Kim et al., “Long-Term Stability of Plasma Oxidized PDMS Surfaces,” Proceedings of the 26th Annual International Conference of the IEEE EMBS San Francisco, Calif., USA, 1-5 Sep. 2004; Makamba et al., “Stable Permanently Hydrophilic Protein-Resistant Thin-Film Coatings on Poly(dimethylsiloxane) Substrates by Electrostatic Self-Assembly and Chemical Cross-Linking” Anal. Chem. 2005, 77, 3971-3978, 20 May 2005; Roman et al. “Surface Engineering of Poly(dimethylsiloxane) Microfluidic Devices Using Transition Metal Sol-Gel Chemistry,” Langmuir 2006, 22, 4445-4451, 25 Mar. 2006; Roman et al., “Sol-Gel Modified Poly(dimethylsiloxane) Microfluidic Devices with High Electroosmotic Mobilities and Hydrophilic Channel Wall Characteristics,” Anal. Chem. 2005, 77, 1414-1422, 1 Mar. 2005; Sharma et al., “Surface characterization of plasma-treated and PEG-grafted PDMS for micro fluidic applications,” Vacuum 81 (2007) 1094-1100, 11 Feb. 2007; Vickers et al., “Generation of Hydrophilic Poly(dimethylsiloxane) for High-Performance Microchip Electrophoresis,” Anal. Chem. 2006, 78, 7446-7452, 5 Oct. 2006; Wang et al., “Modification of poly(dimethylsiloxane) microfluidic channels with silica nanoparticles based on layer-by-layer assembly technique,” Journal of Chromatography A, 1136 (2006) 111-117, 31 Oct. 2006; and Xiao et al., “Surface Modification of the Channels of Poly(dimethylsiloxane) Microfluidic Chips with Polyacrylamide for Fast Electrophoretic Separations of Proteins,” Anal. Chem. 2004, 76, 2055-2061, 25 Feb. 2004.

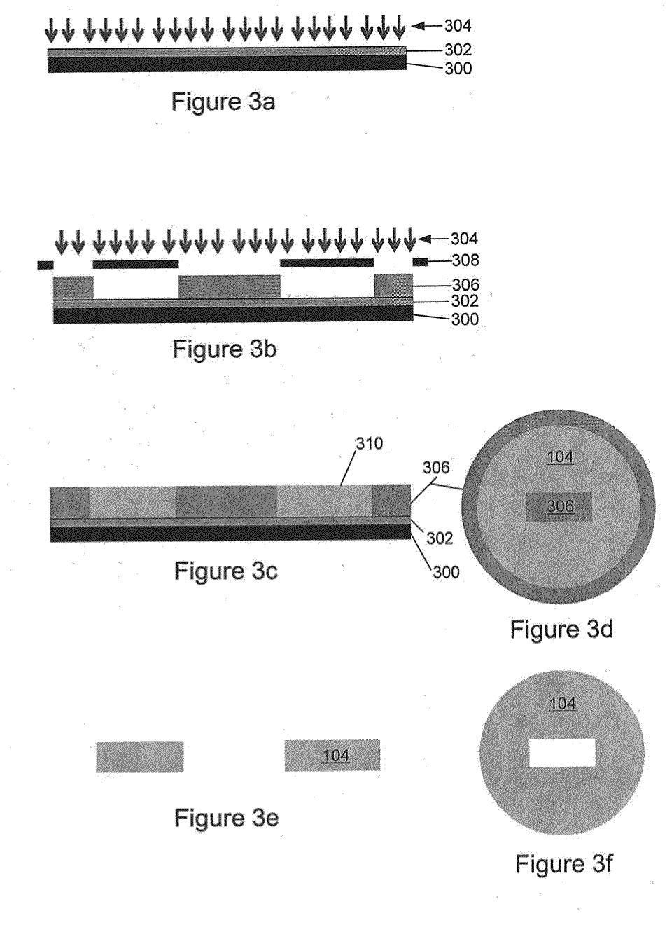

FIGS. 3a through 3f depict an exemplary method for manufacturing the container layer 104 of the skin patch 100 according to various embodiments. The container layer 104 may form at least a portion of the side walls of the container 116 and may be fabricated using a hydrophilic material. To maintain a fixed shape, and a fixed volume, the container layer 104 may be rigid or substantially rigid. One material that may be used to fabricate the container layer 104 is PMMA.

The container layer 104 may be fabricated using similar methods as were used in fabricating the channel layer 102 as discussed in connection with FIGS. 2a-2h. In the depicted embodiments using photolithography techniques to create the container layer 104, the release liner 302 is formed on a wafer 300 using UV light 304 in FIG. 3a. In FIG. 3b, a mask 308 is used during lamination to define the shape of the mold 306 of the container layer 104. In some embodiments, the lamination is repeated twice to produce a vent layer having a thickness of approximately 100 micrometers.

In FIGS. 3c and 3d, a prepolymer mixture 310 is poured into the mold 306. As discussed, the prepolymer mixture 310 may comprise PMMA. When PMMA is used, a curing agent may be mixed with the PMMA in about a 1:100 weight ratio. To prevent bubbles from forming and to release bubbles that do form, the PMMA may be slowly agitated using a stir stick and/or allowed to stand for about 10 minutes. The PMMA may be cured at room temperature for about two hours. After curing, the mold 306 may be peeled or otherwise removed from the container layer 104 as depicted in FIGS. 3e and 3f.

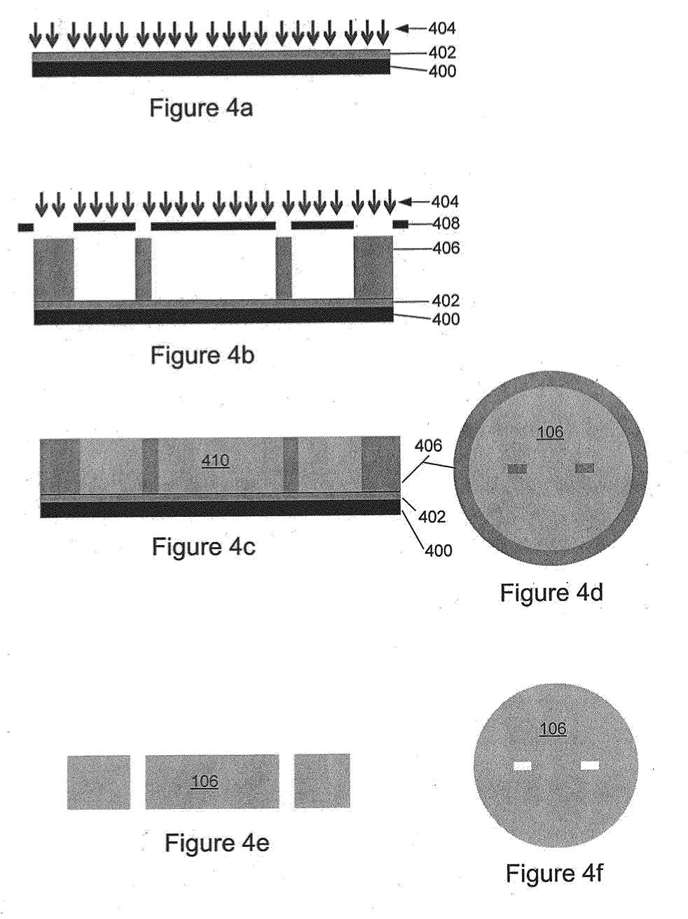

FIGS. 4a through 4f depict a method for manufacturing a vent layer 106 of a skin patch 100 according to various embodiments. The vent layer 106 may form at least a portion of the top wall of the container 116 and may be fabricated using one or more hydrophilic or hydrophobic materials. To limit or prevent evaporation of sweat contained within the container 116 while still providing sufficient fluid flow, the vent layer 106 may include one or more vents 108 in fluid communication with the container 116. The vent layer 106 may be fabricated using PDMS, PMMA, or another suitable material.

The vent layer 106 may be fabricated using similar methods as were used in fabricating the channel layer 102 as discussed in connection with FIGS. 2a-2h. In the depicted embodiments using photolithography techniques to create the vent layer 106, the release liner 402 is formed on a wafer 400 using UV light 404 in FIG. 4a. In FIG. 4b, a mask 408 is used during lamination to define the shape of the mold 406 of the vent layer 106. In some embodiments, the lamination may be repeated ten times to produce a vent layer having a thickness of approximately 500 micrometers. In FIGS. 4c and 4d, a prepolymer mixture 410 is poured into the mold 406. After curing, the mold 406 may be peeled or otherwise removed from the vent layer 106 as depicted in FIGS. 4e and 4f.

In some embodiments, the container layer 104 may be fabricated with the channel layer 102 and/or the vent layer 106. For example, a bi-layer mold may be generated that, when filled, results in a single piece that operates as the channel layer 102 and the container layer 104 or that operates as the container layer 104 and the vent layer 106. The bi-layer mold may be filled with a single material (e.g., PMMA) or may be filled with two or more different materials. To illustrate, when the bi-layer mold is used to generate a single piece that operates as the container layer 104 and the vent layer 106, the mold may first be filled using a hydrophilic material to a first level and then filled using a hydrophobic material between the first level and a second level. The first level may be selected so that the surfaces defining the container 116 are hydrophilic while the surfaces of the vents 108 are hydrophobic. The bi-layer mold may be desirable, for example, in embodiments where an inaccurate alignment of the layers may significantly affect the fluid flow in the skin patch 100.

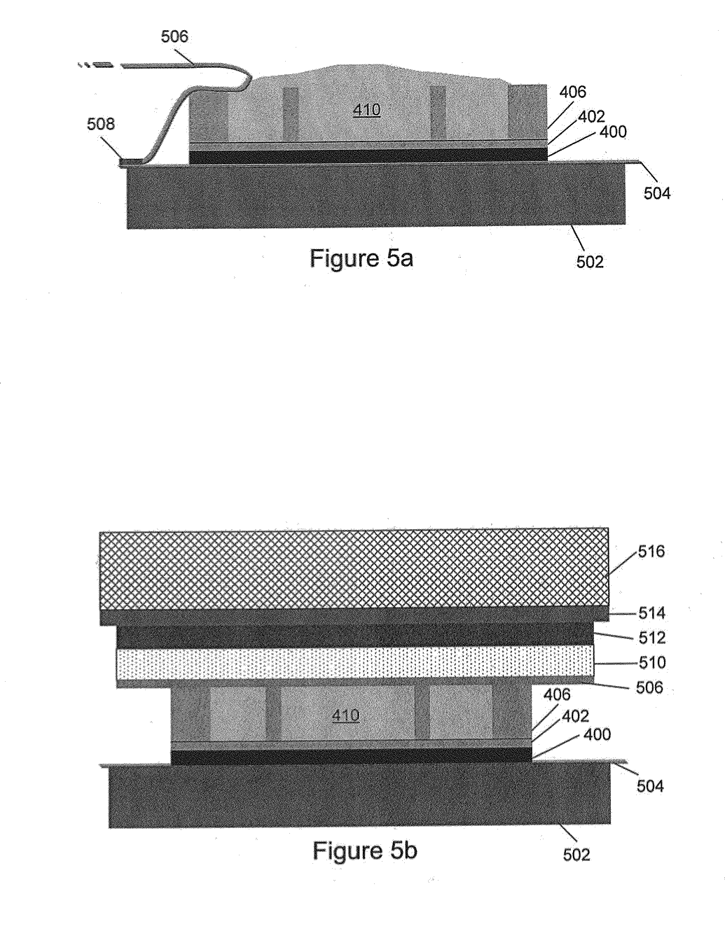

FIGS. 5a and 5b depict a method for molding the various layers according to various embodiments. In some embodiments where the skin patch 100 comprises PDMS and PMMA, the molding process depicted in FIGS. 5a and 5b may be used. The molding method for the channel layer 102, the container layer 104, and the vent layer 106 of the skin patch 100 may be substantially the same in these embodiments.

For the purposes of illustration, the molding technique used in connection with the vent layer 106 is depicted. The wafer 400, release layer 402, and mold 406 filled with a prepolymer mixture 410 may be placed on a metal plate 502. The prepolymer mixture 410 may comprise PMDS or PMMA. After the prepolymer mixture 410 is poured onto the mold 406, a transparent film 506 may be placed over the prepolymer mixture 410. One end of the transparent film may be fixed by tape 508 at one side of the mold 408 as shown in FIG. 5a. The transparent film 506 may be rolled along the top of the mold 406 slowly to prevent bubbles from forming at the interface.

As shown in FIG. 5b, a rigid glass wafer 510 (e.g., a Pyrex® glass wafer), a rubber sheet 512, metal plate 514, and weight block 516 may be stacked sequentially to form a compression mold. One technique for doing so is described by B-H et al., “Three-dimensional micro-channel fabrication in polydimethylsiloxane (PDMS) Elastomer,” J. Microelectromech. Syst. Vol. 9 pp 76-81, 2000. The compression mold may be heated on the hotplate for curing (e.g., in embodiments where the prepolymer mixture 410 comprises PDMS). For PDMS, the curing time may be about 30 minutes at about 150° C. In embodiments where one or more of the layers formed by a mold (e.g., mold 406) is thicker than about 500 micrometers, a lower temperature and a longer time for curing are used to avoid cracking of the mold. In one embodiment, the curing time may be about three hours at about 100° C. In embodiments where the prepolymer mixture 410 comprises PMMA, the PMMA may be cured at the room temperature for about two hours.

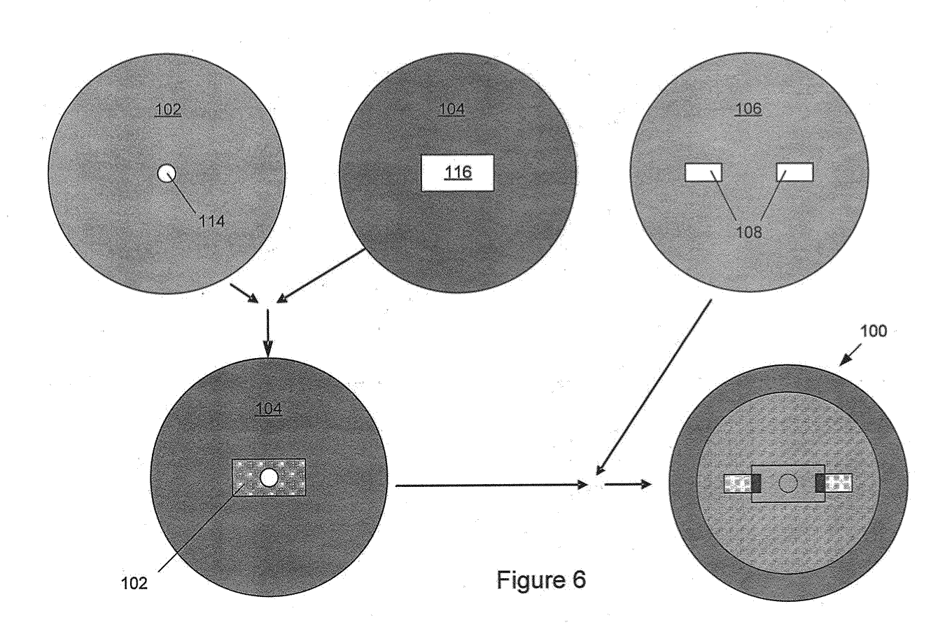

FIG. 6 depicts a flow diagram for assembling the various layers according to the exemplary methods. Prior to assembly, one or more of the layers may be coated, shaped, or otherwise modified. In some embodiments, surfaces that define the container 116 may be coated with a hydrophilic material. For example, the channel layer 102 may be coated with a hydrophilic material along its top surface and along the interior of the opening 114. The bottom surface of the vent layer 106 may also be coated with a hydrophilic material. In some embodiments, the surfaces that define the container 116 and/or one or more electrodes in contact with the container 116 may be coated with an enzyme that reacts with the glucose in the sweat (e.g., glucose oxidase).

In certain embodiments, components comprising a volume indicator may be disposed in the container 116 for indicating if the predetermined volume of sweat has been collected. As discussed herein, the volume indicator may comprise two or more electrodes in contact with the container 116 that are connected to two or more electrodes on a top surface of the vent layer 106. The volume indicator may also be optical, chemical, mechanical, or the like.

The channel layer 102, the container layer 104, and the vent layer 106 may be assembled in any number of ways. In the embodiment shown, the channel layer 102 and the container layer 104 are first aligned and bonded together. The alignment may be performed using a stereomicroscope or be performed automatically. In some embodiments and as shown, the opening 114 and the container 116 are shaped such that the alignment step may be skipped. The channel layer 102 and the container layer 104 may be bonded together using a urethane or an acrylic adhesive at room temperature. Other adhesives may alternatively or additionally be used.

After the channel layer 102 and the container layer 104 are bonded together, the vent layer 106 may be bonded to the opposite surface of the container layer 104. Prior to bonding, the vent layer 106 may be aligned with the container 116 such that the vents 108 overlap, or partially overlap, the container 116. In some embodiments, the container 116 and/or the vents 108 may be symmetrically positioned and/or shaped such that the alignment step can be skipped. In other embodiments, the container layer 104 and the vent layer 106 may be manufactured as a single layer. A urethane adhesive and/or an acrylic adhesive may be used to bond the container layer 104 to the vent layer 106 at room temperature. Other bonding techniques or adhesives may also be used.

Although examples of methods of making a skin patch 100 have been described, it is understood that alternative or additional embodiments will be apparent to those skilled in the art. Further, it should be noted that the skin patch 100 may be fabricated using materials other than those specified here. The above disclosure is not intended to limit the scope of the present application.

### Methods of Use

The skin patch 100 may be used by a diabetic patient to collect sweat to measure his or her glucose level. The skin patch 100 may replace a finger stick or other methods of drawing blood. To use, the patient attaches the skin patch 100 to a target location on the surface of the skin. When the skin patch 100 has collected a sufficient volume of sweat, the patient may use a measurement device to quantitatively measure the sweat glucose level. The patient, based on the sweat glucose level or a blood glucose level that corresponds to the sweat glucose level, may self-administer insulin as needed. Prior to use, the patient may clean an area of skin to remove residual glucose present at the skin surface. Exemplary wipes that may be used are described in U.S. Patent Publication No. US 2003/0176775 A1 filed Feb. 4, 2003 and entitled “Cleaning Kit for An Infrared Glucose Measurement System.” For example, the patient may use a wipe impregnated with a cleanser that does not interfere with glucose detection and/or a surfactant that modifies one or more properties of the sweat and/or the skin surface (e.g., sodium lauryl sulfate (SLS)). In some embodiments, the wipe may contain a chemical marker that is identifiable by a measurement device to confirm that the skin was wiped before the sweat was collected in the skin patch 100. In certain embodiments, the wipes may contain a marker used to detect when the container 116 is filled. For example, the wipe may comprise a reactant that reacts with another chemical within the container 116 to indicate (e.g., via a color change) that the container 116 is filled.

The skin patch 100 may be attached to the surface of the skin in a number of ways. In some embodiments, the patient may remove a release liner from the bottom surface of the channel layer 102 to expose a pressure-sensitive adhesive that may adhere to the skin. In other embodiments, other adhesives may be used such as heat-sensitive or soluble adhesives. Alternatively, the skin patch 100 may be positioned using an elastic band configured to hold the skin patch 100 in place. In other embodiments, the patient may tape the skin patch 100 to the surface of the skin using, e.g., medical tape, or may hold the skin patch 100 to the surface of the skin.

To determine when the predetermined volume is collected, the patient may consult a volume indicator. The volume indicator may be integrated into the skin patch 100 or may be interrogated by another device, such as a measurement device. In some embodiments, the patient may simply remove the skin patch 100 after a certain length of time, for example, one minute, two minutes, five minutes, or ten minutes.

After the predetermined volume is collected, the skin patch 100 may be interrogated using a measurement device. In some embodiments, the measurement device may be placed in contact with the skin patch 100 at one or more electrodes. In other embodiments, the skin patch 100 may be removed from the skin and inserted into, or otherwise contacted with, the measurement device. The skin patch 100 may be single-use only.

### Measurement Device

As discussed above, a measurement device may be used to measure the amount of glucose in the sweat collected by the skin patch 100. In some embodiments, the measurement device may interrogate the skin patch 100. The device measures the total quantity of glucose present in a fixed volume, and then converts the glucose measurement into a sweat glucose or blood glucose concentration. In general, the measurement device typically comprises a display, to display data. The device may also include warning indicators (e.g., a word prompt, flashing lights, sounds, etc.) to indicate that a patient's glucose levels are dangerously high or dangerously low. In addition, as described briefly above, the measurement device may also be configured to verify that a skin-cleaning procedure has been performed. For example, when wipes with a marker have been used, the marker remains on the skin surface. If the measurement device detects the marker, then the measurement proceeds. If the measurement device does not detect the marker, the measurement does not proceed. In one variation, the measurement device provides an indication to the user, that the skin surface must be cleaned prior to use (e.g., using a word prompt, colored and/or flashing lights, and/or various sounds).

In some embodiments, the measurement device may be configured to estimate sweat flux. It may be desirable to use the sweat flux estimate to correct the sweat glucose measurement or to flag sweat collections that are above or below acceptable limits. Sweat flux is generally defined as the flow rate of the sweat. Sweat flux may vary in the presence of heat, stress, diaphoretic drugs, or other stimulus. For example, the amount of time from when the container 116 is about 10% full to when it is full may be measured to determine sweat flux. In these embodiments, the skin patch 100 (or a skin patch holder configured to hold a skin patch 100 at the surface of the skin) may comprise additional fill sensing and timing circuits.

The configuration of the measurement device is dependent on the configuration of the skin patch. For example, when the measurement device is to be used with a skin patch 100 having electrodes, the measurement device provides an electrical contact with the interface layer, and is either powered by the electrical contact, or is powered by an independent power source (e.g., a battery within the patch itself, etc.). The measurement device also typically comprises a computer processor to analyze data. Conversely, when the measurement device is configured for optical detection, the measurement device is configured to provide optical contact or interaction with the skin patch 100. In this variation, the measurement device also typically comprises a light source. In some variations, the measurement device comprises both the necessary electrical contacts and the necessary optics so that a single measurement device may be used with a patch having various configurations of patch layers.

The measurement device may further comprise computer executable code containing a calibration algorithm, which relates measured values of detected glucose to blood glucose values. For example, the algorithm may be a multi-point algorithm, which is typically valid for about 30 days or longer. For example, the algorithm may necessitate the performance of multiple capillary blood glucose measurements (e.g., blood sticks) with simultaneous patch measurements over about a one day to about a three day period. This could be accomplished using a separate dedicated blood glucose meter provided with the measurement device described herein, which comprises a wireless (or other suitable) link to the measurement device. In this way, an automated data transfer procedure is established, and user errors in data input may be minimized.

Once a statistically significant number of paired data points have been acquired having a sufficient range of values (e.g., covering changes in blood glucose of about 200 mg/dl), a calibration curve may be generated, which relates the measured sweat glucose to blood glucose. Patients can perform periodic calibrations checks with single blood glucose measurements, or total recalibrations as desirable or necessary.

The measurement device may also comprise a memory for saving readings and the like. The measurement device typically comprises a processor configured to access the memory and execute computer executable code stored therein. It should be understood that the measurement device may include other hardware such as an application specific integrated circuit (ASIC). In addition, the measurement device may include a link (wireless, cable, and the like) to a computer. In this way, stored data may be transferred from the measurement device to the computer, for later analysis, etc. The measurement device may further comprise various buttons, to control the various functions of the device and to power the device on and off when necessary.

### Kits

Also described here are kits. The kits may include one or more packaged skin patches, either alone, or in combination with other skin patches, a measurement device, and/or instructions. In one variation, the kits comprise at least one patch having a volume indicator. Typically the skin patches are individually packaged in sterile containers or wrappings and are configured for a single use.

What is claimed is:

1. A device comprising:
a channel layer configured to direct sweat that has come to the skin surface to an opening;
a container layer in fluid communication with the opening and defining at least a portion of a container configured to contain a volume of less than about one microliter of the sweat; and
a vent layer comprising a vent adjacent to the container.

2. The device of claim 1, wherein the channel layer comprises a plurality of channels, each of the channels in fluid communication with the opening.

3. The device of claim 1, wherein the channel layer defines at least a portion of a bottom side of the container.

4. The device of claim 3, wherein the channel layer comprises an electrode in contact with the container.

5. The device of claim 1, wherein the opening has a diameter of less than about seven hundred micrometers.

6. The device of claim 1, wherein the volume of the container is fixed.

7. The device of claim 1, wherein the container layer comprises an electrode in contact with the container.

8. The device of claim 1, wherein the vent is hydrophobic.

9. The device of claim 1, wherein the vent is configured to reduce evaporation from the container.

10. The device of claim 1, wherein an external surface of the vent layer comprises external electrodes configured to contact a measurement device, each of the external electrodes connected to an internal electrode in contact with the container.

11. The device of claim 1, wherein the vent layer has a thickness of approximately 500 micrometers.

12. The device of claim 1, wherein the vent layer defines at least a portion of a top side of the container.

13. The device of claim 1, wherein the vent layer comprises one or more electrodes in contact with the container.

14. The device of claim 1, wherein the channel layer comprises a mechanism to induce the sweat.

15. The device of claim 1, wherein the container comprises glucose oxidase.

16. The device of claim 1, wherein the container comprises a dye.

17. The device of claim 1, wherein the device can be used with a measurement device when the container contains the volume of the sweat.

18. The device of claim 1, wherein the channel layer has a thickness of about 200 micrometers.

19. The device of claim 1, wherein the container layer has a thickness of about 200 micrometers.

20. The device of claim 1, wherein the container layer defines a side portion of the container.

21. A method for measuring glucose comprising:
collecting a predetermined volume of sweat from skin using a skin patch, wherein the volume is less than about one microliter of sweat; and
measuring an amount of glucose in the volume of the sweat.

22. The method of claim 21, further comprising stimulating sweat production.

23. The method of claim 21, further comprising determining whether the volume of the sweat is adequate prior to measuring the amount of the glucose.

24. The method of claim 21, wherein measuring the amount of the glucose comprises contacting the skin patch with a measurement device.

25. A kit comprising:
a skin patch configured to collect a predetermined volume of sweat, wherein the predetermined volume is less than about one microliter; and
a measurement device configured to measure an amount of glucose in the sweat, wherein the measurement is based on the predetermined volume.

26. The kit of claim 25, wherein the skin patch comprises a container configured to contain the predetermined volume.

27. The kit of claim 26, wherein the container is configured to retain its shape if the skin patch is deformed.

28. The kit of claim 25, wherein the skin patch comprises at least two electrodes in contact with the container.

29. The kit of claim 25, wherein the skin patch is configured to provide an indication if the predetermined volume is collected.

30. The kit of claim 25, wherein the skin patch is configured for single use.

31. The kit of claim 25, further comprising a plurality of the skin patches.

32. The kit of claim 25, wherein the measurement device comprises at least two electrodes configured to contact the skin patch.

33. The kit of claim 25, wherein the measurement device comprises an inlet configured to receive the skin patch.
